(19) United States
(12) Patent Application Publication
(10) Pub. No.: US 2007/0196367 A1
(43) Pub. Date: Aug. 23, 2007

(54) Methods of preventing and treating Alzheimer's disease, age related macular degeneration and other diseases involving extra-cellular debris through the inhibition of the complement system

(76) Inventor: Valentin Dinu, Manchester, CT (US)

Correspondence Address: VALENTIN DINU, 465 BUCKLAND HILLS DRIVE, APT. 35234, MANCHESTER, CT, 06042, US

(21) Appl. No.: 11/708,271

(22) Filed: Feb. 20, 2007

Related U.S. Application Data
(60) Provisional application No. 60/775,923, filed on Feb. 22, 2006.

(51) Int. Cl. A61K 39/395; A61K 31/7088; A61K 38/12

(52) U.S. Cl. 424/141.1; 424/130.1; 514/2; 514/44; 514/9

(57) ABSTRACT

This invention proposes that the best therapeutic strategy for treating and/or preventing Alzheimer's disease (AD), age related macular degeneration (AMD) and other diseases that exhibit extra-cellular debris deposits, such as atherosclerosis, is the inhibition of the complement pathway. A model for the accumulation of extra-cellular debris through the activation of the complement pathway is presented, and the primary pathogenic role of the debris in the etiology of the disorders is explained. Previously identified complement inhibitors are identified as therapeutic agents for the treatment and/or prevention of AD, AMD and other diseases that exhibit extra-cellular debris deposits.

### CROSS-REFERENCE TO RELATED APPLICATIONS

This application claims the benefit of PPA Appl. No. 60/775,923, filed Feb. 22, 2006 by the present inventor.

### FEDERALLY SPONSORED RESEARCH

Not Applicable

### SEQUENCE LISTING OR PROGRAM

Not Applicable

### BACKGROUND OF THE INVENTION

1. Field of the Invention

This invention relates to a method for preventing and treating Alzheimer's Diseases, Age Related Macular Degeneration, and other diseases involving the gradual accumulation of extra-cellular debris, such as atherosclerosis or glomerulonephritis. More particularly, this method relies on the inhibition of the complement pathway to prevent the formation of extra-cellular debris that causes the breakdown of the normal biological function of surrounding tissue. This invention also relates to a method for detection of predisposition for and/or early detection of the disorders through identifying complement-pathway related genomic polymorphisms and biomarkers that are associated with the disorders.

2. Prior Art

Alzheimer's disease (AD) is a brain disorder that seriously affects a person's ability to carry out daily activities. The disease usually begins after age 60, and risk goes up with age. While younger people also may develop AD, it is much less common. About 5 percent of men and women ages 65 to 74 have AD, and nearly half of those age 85 and older may have the disease. It is estimated that more than 4 million people in the USA have AD. There is no known cure for AD.

Age Related Macular Degeneration (AMD) affects the central region of the retina, known as the macula, and is the main cause of blindness in elderly Americans and persons of Western European descent. It is estimated that there are more than 1 million people in the USA alone with advanced forms of AMD, and more than 7 million with the intermediate form of AMD (National Eye Institute website). There is no known cure for AMD.

The hallmark of AD, AMD, and other diseases, such as atherosclerosis or glomerulonephritis (GN), is the formation of extra-cellular deposits in the vicinity of the affected tissues (neurons for AD, photoreceptors for AMD, arteries for atherosclerosis, glomeruli in kidney for GN). These deposits are known as amyloid or senile plaques in the case of AD, drusen (“stony nodule” in German) for AMD, plaque for atherosclerosis, drusen or deposits in some types of GN. The extra-cellular debris-like deposits form over time, often over many years or decades, as is the case with drusen in AMD. The physical appearance, size and shape of the deposits varies across diseases and affected tissues, e.g. plaques on the artery walls in atherosclerosis, spherical or irregular shaped in AMD or GN, and irregular in AD. The composition of these deposits also varies across diseases and affected tissues, but generally includes complement components, other immune related molecules, cholesterol, apolipoprotein E, amyloid beta, etc.

Current Therapeutic Approaches for AD and AMD

Currently, there are no known treatments that can stop AD. For some people in the early and middle stages of the disease, however, the drugs tacrine (Cognex®), donepezil (Aricept®), rivastigmine (Exelon®), or galantamine (Razadyne®, formerly known as Reminyl®) may help prevent some symptoms from becoming worse for a limited time. Another drug, memantine (Namenda®), has been approved to treat moderate to severe AD, although it also is limited in its effects. Epidemiological evidence indicates that extended use of nonsteroidal anti-inflammatory drugs (NSAIDs) results in a reduced risk of AD (Broe, Grayson et al. 2000; Etminan, Gill et al. 2003; Szekely, Thorne et al. 2004). After the onset of the disease, however, there is no effective treatment.

In the case of AMD, recent drug therapies have focused on targeting the VEGF pathway with the aim of reducing neovascularization, present in the worse form of the disease (neovascular or wet AMD). Macugen is an FDA approved drug that inhibits abnormal blood vessel growth by blocking VEGF, a protein that causes promotes vessel growth. Macugen is not a cure, but it can help to slow further vision loss and also to help preserve what vision one has left (Pfizer website). Other potential treatments for wet AMD that are under investigation include anti-VEGF antibodys, anti-VEGF aptamer (NX-1838), and PKC412, an inhibitor of protein kinase C. Cytochalasin E (Cyto E), a natural product of a fungal species that inhibits the growth of new blood vessels is also being investigated to determine if it will block growth of abnormal blood vessels in human eyes (Udagawa, Yuan et al. 2000). The role of hormone replacement therapy is being investigated for treatment of AMD in women (women are at higher risk of developing AMD).

Extra-cellular Debris Deposits

With age, drusen accumulates in the eye between the retinal pigment epithelium (RPE) and Bruch's membrane (BrM). Drusen can be observed during a funduscopic eye exam (an examination in which the pupil is dilated, so that the ophthalmologist can look into the eye and see the structures at the back of the eye). Drusen can have various sizes and shapes. Generally, the higher the macular area covered by drusen, the higher the risk of developing AMD (Davis, Gangnon et al. 2005). Normal eyes may have maculas free of drusen, yet drusen may be abundant in the periphery of the retina (Hageman, Mullins et al. 1999). For over a century, researches have described drusen and proposed models for its origin and role in AMD—excellent recent reviews are provided by Hageman et al (Hageman, Luthert et al. 2001) and Penfold et al (Penfold, Madigan et al. 2001). Briefly, various potential sources for the origin of drusen have been named: RPE, BrM, choroid, photoreceptors, etc.; and various causative processes have been proposed, such as oxidative stress, photoreceptor phagocytosis, immune response involving macrophages, cytokines, and/or the complement system, etc.

Recently, several groups have identified the major components of drusen (Hageman, Mullins et al. 1999; Crabb, Miyagi et al. 2002; Johnson, Leitner et al. 2002; Dentchev, Milam et al. 2003; Anderson, Talaga et al. 2004; Howes, Liu et al. 2004; Curcio, Presley et al. 2005; Curcio, Presley et al. 2005). Drusen are rich in complement relate proteins, such as clusterin, vitronectin, and terminal complement complexes (C5b-C9), as well as cholesterol, lipofuscin, apolipoprotein E, advanced glycation end products (AGEs), amyloid P, amyloid beta, etc. Many of the molecules associated with drusen are also found in pathologic deposits encountered in other diseases, such as AD, glomerulonephritis, and atherosclerosis, raising the possibility of common disease pathways.

Results from recent genetic association studies have identified a non-synonymous mutation in complement factor H (CFH), as providing extra susceptibility for developing AMD (Edwards, Ritter et al. 2005; Haines, Hauser et al. 2005; Klein, Zeiss et al. 2005). CFH is an inhibitor of the alternative complement pathway (one of the three currently known branches that lead to complement activation). Other studies have explored the possible involvement of other complement pathway components, such as BF, C2, MBL2, and C7 in the etiology of AMD (Gold, Merriam et al. 2006), (Dinu, Miller et al. in press).

To date, there is no commonly accepted model for the origin of drusen or for its role in the etiology of AMD. Inflammation and the role of the immune system in the pathology of AMD is increasingly being recognized (Hageman, Luthert et al. 2001; Johnson, Leitner et al. 2001; Anderson, Mullins et al. 2002). The hypotheses for the etiology of AMD, however, tend to name drusen formation as a secondary factor that contributes to AMD following the effects of some primary pathogens, which can be genetic or environmental (Anderson, Mullins et al. 2002; Yoshida, Ohno-Matsui et al. 2005).

The method for treating and/or preventing disorders such as AMD and AD presented here is based on a novel model that I propose for the etiology of AD and AMD, drawn from observations across multiple diseases, and that goes against the present common belief in the medical and scientific community. This model identifies as the primary pathologic event in the etiology of AD and AMD the elimination via exocytosis (emission of membrane vesicles) of the terminal membrane attack complex (MAC, a complex including complement components C5b, C6, C7, C8 and multiple C9) of the complement system from cells where the complement system is sub-lethally activated.

The Complement System

The complement system is part of the innate immune system, and is directed against micro-organisms and infected cells. It can also, however, affect normal “bystander” cells—a process which has been defined as autotoxicity (McGeer and McGeer 2002). The immune roles of the complement system include: recognition of target cells, opsonization (the process by which cells are marked for phagocytosis), inflammatory stimulation, and cell lysis through the insertion of the membrane attack complex (MAC) into cell membranes (McGeer and McGeer 2002). The complement system is composed of soluble blood plasma proteins and cell membrane proteins. A diagram of some of the known complement proteins is presented in FIG. 1. Bohana-Kashtan et al (Bohana-Kashtan, Ziporen et al. 2004) provides an excellent recent review of the complement components and the cell signals transduced by the complement. Briefly, the complement cascade can be activated through one of the three currently known pathways: classical, alternative, and lectin. The classical complement pathway is activated when complement component C1q binds to a target either directly or via an antibody-antigen complex. The binding of C1q activates a cascade of proteases (C1r, C1s, C4, C2, and C3), which amplifies the original response. The alternative complement pathway is activated directly by the hydrolysis of C3. The lectin pathway is initiated by binding of mannan-binding lectin to sugar residues on bacterial cell walls. Cleavage products C4b and C3b (formed by proteolytic cleavage of C4 and C3, respectively) attach to exposed sites on target cells located near the C1q binding site. The attached fragments then become ligands for complement receptors on phagocytes, initiating the process of opsonization. The cleavage fragments C3a, C4a, and C5a, known as anaphylotoxins, stimulate inflammation by attracting phagocytes to the region. If the complement system is fully activated, it proceeds to sequentially assemble the terminal components (C5b, C6, C7, C8, and multiple copies of C9) into the MAC, a transmembrane complex that can cause cell lysis (McGeer and McGeer 2002) (FIG. 1).

To protect against autotoxicity, the complement system is kept in strict control by a large number of complement regulatory molecules, which include C1 inhibitor, CFH, Complement Factor I, C4b binding protein, CD35, CR1/CD46, CD46/MCP, decay accelerating factor (DAF/CD55). Formation of the MAC (C5b-9 terminal complex) is inhibited by at least 3 proteins (McGeer and McGeer 1998). Vitronectin (S-protein) and clusterin (apolipoprotein J) bind to the soluble C5b-7 complex blocking its insertion into the membrane (Murphy, Saunders et al. 1989). Protectin (CD59) binds to C8 and C9 and inhibits the incorporation and subsequent polymerisation of further C9 (Rollins, Zhao et al. 1991).

The formation of the MAC in the cell membrane can lead to cell lysis and death. Cells undergo swelling accompanied by an influx of Ca²⁺. It has been shown, however, that nucleated cells have the ability to eliminate the potentially lethal C5b-9 complex by endocytosis and/or exocytosis (Morgan 1988; Morgan, Daniels et al. 1988; Morgan 1989). This is the process that is identified here as the primary pathologic event in the etiology of AD and AMD. The inhibition of the complement pathway is presented as the best method to prevent and/or cure AMD, AD and other diseases involving extra-cellular debris, like atherosclerosis.

Uses of Complement Inhibition for Diseases Other than AD, AMD and Atherosclerosis

The use of complement inhibition has been proposed before in the context of other diseases.
U.S. Pat. No. 4,686,100 proposes inhibiting C5a via an antibody to treat adult respiratory distress syndrome. I propose the use of complement inhibitors in AD, AMD and other disorders exhibiting extra-cellular debris.
U.S. Pat. No. 5,135,916 claims the inhibition of the terminal complex MAC (C5-9) using a number of endogenous proteins or antibodies. The authors, however, claim the use of such inhibition in inflammatory responses and to inhibit cell activation and cytolysis. I claim here in my model that the complement system activation leads to AD and AMD not by cytolysis but by the gradual elimination and accumulation of cellular debris from cells where the complement system is sub-lethally activated. I propose that this debris deposition happens before the local inflammation develops, and actually that the debris deposition leads to inflammation. The authors did not mention AD or AMD as applications, and they focused only on the MAC inhibition and with a limited number of inhibiting methods, while I believe that other methods of MAC inhibition as well as the inhibition of the complement cascade at other stages will be effective as well.
U.S. Pat. No. 5,472,939 proposes the inhibition of the complement system by using the endogenous inhibitor CR1, and the authors mention its uses in reducing inflammation and myocardial infarct. They did not mention AD or AMD, and they focus on inflammation reduction, while I propose the inhibition of the complement prior to the development of local inflammation
U.S. Pat. No. 5,506,247 proposes the inhibition of the complement system by interrupting the processing of the C5 protein using compounds with an aromatic ring. The authors did not identify AD or AMD as targeted diseases. I propose that inhibition at other stages of the complement cascade can be effective as well.
U.S. Pat. No. 5,573,940 proposes the inhibition of the terminal MAC by introducing the MAC inhibitor CD59 into the cells with the aim of protecting transplanted organs and cells from cytolysis. I propose that inhibition at other stages of the complement cascade can be effective as well, not only the terminal MAC. I propose the use in AD, AMD and other disorders exhibiting extra-cellular debris. I propose the inhibition of the sub-lethally activated complement system in AD and AMD to prevent debris elimination in the first place, rather than the effect of cell destruction via cytolysis.
U.S. Pat. No. 5,635,178 proposes the use of monoclonal antibodies to inhibit the MAC (C5-9) formation to prevent cell activation. I propose the complement inhibition specifically in AD, AMD and other disorders exhibiting extra-cellular debris. I propose the inhibition of the complement at other stages as well and through other methods as well not only antibodies. I propose that the main role of the inhibition is to prevent the elimination of the MAC from cells where the complement system is sub-lethally activated with the goal of preventing the deposition of extra-cellular debris.
U.S. Pat. No. 5,660,825 claims the inhibition of the terminal MAC using proteins and antibodies for the treatment of autoimmune disorders and other complement-mediated disease states. I propose the use of complement system inhibition in AD and AMD specifically, and I propose the use of various methods to inhibit the complement system at different stages, including the MAC.
U.S. Pat. No. 6,090,777 proposes the use of C1-esterase inhibitor as therapeutic or prophylactic treatment method of acute myocardial infarction. I propose the use of complement inhibitors in AD, AMD and other disorders exhibiting extra-cellular debris.
U.S. Pat. No. 6,248,365 proposes the use of complement inhibitors, especially of C1 inactivator or of factors I or H, for the prophylaxis and therapy of chronic inflammatory intestinal disorders, inflammatory skin disorders and purpura. I propose the use of complement inhibitors in AD and AMD.
U.S. Pat. No. 6,355,245 proposes the use of anti-C5 antibodies, e.g., monoclonal antibodies, to treat glomerulonephritis. While I give glomerulonephritis as an example of related disease, my focus is on AMD and AD. The authors did not identify AD or AMD as targets of complement system inhibition.
U.S. Pat. No. 6,538,028 proposes inhibiting the complement activation by the administration of platelet activity modulator, with particular applications for a transplanted tissue. I propose the inhibition of the complement system in AD and AMD.
McGeer and McGeer (McGeer and McGeer 1998) proposed in 1998 that in the future, complement inhibitors could be used to treat central nervous system diseases. The authors recounted the protective effects of such inhibitors in a series of animal models of immune disorders, particularly transplant rejection and ischemia-reperfusion injury, and proposed that similar effects could be achieved for nervous system disorders as well. But the authors did not identify AD as being caused specifically by the effects of the complement system, and namely by the elimination of MAC from cells where the complement was sub-lethally activated. The authors only observed that the complement system is involved in the pathology of various neurological diseases, and not the primary cause of the disease, and proposed that in the future complement inhibitors might prove effective in their treatment. We, however, identify the mechanism that leads to the development of AD and AMD through the gradual elimination of complement debris as being the primary pathologic event that leads to the development of the disease. And I propose that AD and AMD can be prevented and treated by targeting and opposing this particular process of MAC elimination and accumulation of extra-cellular debris.

### SUMMARY

This invention proposes that the best therapeutic strategy for countering AD, AMD and other diseases that exhibit extra-cellular debris deposits would target the inhibition of the complement pathway. In a preferred embodiment, the inhibition of the complement cascade would be early/preventive (e.g., before the full onset of a heterogeneous immune response and inflammation that would be harder to control), local (e.g., targeted for the eye, or even RPE, in the case of AMD), and modulated to the individual's needs (e.g., based on the general activation state of the immune system in a certain individual, based on genetic and epigenetic characteristics, age, etc). Alternatively, the inhibition of the complement cascade can be organism wide or localized to the affected and surrounding tissues. The inhibition can be performed at any stage of the complement cascade, or focused on a particular step, such as the inhibition of the complement terminal membrane attack complex (MAC), formed by proteins C5b, C6, C7, C8, and multiple C9. To achieve the prevention and/or cure of the disorders, one ore more inhibitors of the complement pathway can be used, such as small molecules, antibodies, proteins or peptides, microRNAs and siRNAs.

### BRIEF DESCRIPTION OF THE DRAWINGS

FIG. 1. Complement pathway from KEGG database

FIG. 2. Role of complement system in drusen formation and proposed targets of therapeutic interventions through the inhibition of the complement pathway (bottom).

### DETAILED DESCRIPTION OF THE INVENTION

My method is based on a novel model that I propose for the etiology of Alzheimer's Diseases (AD) and Age Related Macular Degeneration (AMD), drawn from observations across multiple diseases, and that goes against the present common belief in the medical and scientific community. My model identifies as the primary pathologic event in the etiology of AD and AMD the elimination via exocytosis (emission of membrane vesicles) of the terminal membrane attack complex (MAC, C5-9) of the complement system from cells where the complement system is sub-lethally activated. The accumulation of complement debris at a rate that exceeds the rate of debris removal, e.g. by macrophages, gradually leads to a series of events that culminate in the development of disease. Following the “seeding” by complement debris, other molecules, such as amyloid-beta, apolipoprotein E, etc., accumulate gradually and the debris deposits start growing in size and quantity. A feedback pro-inflammatory cycle is generated where the existing cellular debris engages other immune components, e.g. dendritic cells, macrophages and neutrophils, as well as further activation of the complement. The resulting inflammation affects the surrounding tissues (e.g. photoreceptors and retinal pigment epithelium in the case of AMD, or neurons in the case of AD) and leads to disease.

MAC Elimination and Extra-cellular Debris Formation as Primary Pathologic Event

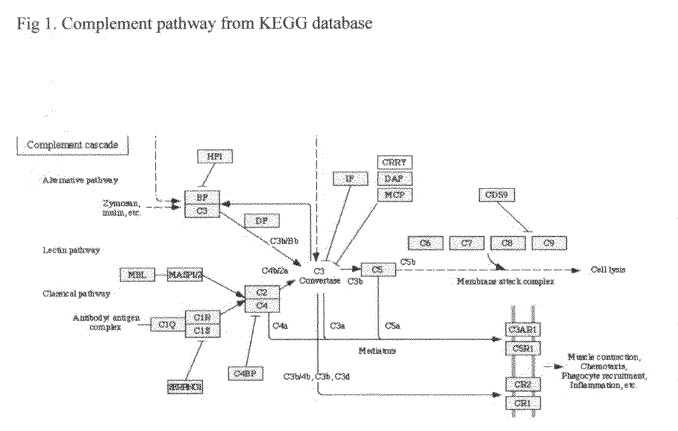

I propose that the formation of drusen is a primary pathologic event that leads to the development of AMD, AMD and other disorders exhibiting extra-cellular debris, such as atherosclerosis. Further, I propose that a central role in the accumulation of drusen is played by the sub-lethal activity of the complement system. Assembly of the terminal membrane attack complex (MAC), composed of 5 complement proteins—C5b, C6, C7, C8 and multiple C9—produces a trans-membrane protein channel that inflicts damage to cells (FIG. 1). As a protective measure, cells eliminate the MAC from their surface either by exocytosis (emission of membrane vesicles) or endocytosis. I propose that the formation of drusen is seeded by the elimination and accumulation of debris from cells (such as neurons in AD or RPE in AMD) in which the complement was sub-lethally activated.

Such a model would account for the fact that 3 out of the 5 most common proteins encountered in both normal and AMD drusen are MAC related: clusterin, vitronectin, and C9 (Crabb, Miyagi et al. 2002). The activation of the complement system together with the extra-cellular debris then triggers a local pro-inflammatory feedback loop involving dendritic cells, neutrophils, macrophages for clearing the debris, as well as cell adhesion and proliferation. Following the “seeding” of drusen by complement debris, a number of other molecules are entrapped and accumulate in the drusen areas. Such molecules include apolipoprotein E, amyloid beta, crystallin, lipofuscin, etc. The growing heterogeneous deposits provide further local pro-inflammatory stimuli. Gradually, the region affected increases in size, affecting the normal functioning of the surrounding tissues. A more detailed support for the drusen formation model based on results from literature is given below.

Literature Support for Drusen Formation Model
1. Proteomic results. Recently, more than 120 drusen proteins have been identified (Crabb, Miyagi et al. 2002). Three of the top five most common drusen proteins are MAC-related: clusterin, vitronectin, and C9. C9 is the most abundant subunit of MAC (up to 18 C9 molecules polymerize in one MAC); clusterin and vitronectin bind to the soluble C5b-7 complex blocking its insertion into the membrane (Murphy, Saunders et al. 1989). The origin of clusterin and vitronectin in drusen could be from their binding to the forming MAC complex prior to its full formation and insertion in the cell membrane, or later, in the extra-cellular debris.
2. Electron microscopy findings. Two decades ago, Ishibashi et al (Ishibashi, Patterson et al. 1986) observed and described a 4-stage process of “budding” of RPE cells: “By electron microscopy, the progression of drusen formation could be classified into four stages. Stage I showed budding or evagination of retinal pigment epithelial cells into the subpigment epithelial space. This evaginated portion was connected to the retinal pigment epithelial cell cytoplasm and was surrounded by its basement membrane. In Stage II the evaginated portion of the cell was completely separate from the cytoplasm of its parent retinal pigment epithelial cell. In Stage III, the evaginated portion showed degeneration and disintegration. Finally, in Stage IV, an accumulation of vesicular, granular, tubular, and linear material was seen free within the nodular space beneath the retinal pigment epithelial cell.” (Ishibashi, Patterson et al. 1986) The process described above is consistent with the process of MAC exocytosis.
3. Genomic results. Recent studies of genomic association with AMD identified a non-synonymous mutation, Y402H, in the CFH gene, an inhibitor of the complement pathway (Edwards, Ritter et al. 2005; Haines, Hauser et al. 2005; Klein, Zeiss et al. 2005). The authors estimated that the mutation increases the risk of AMD at least 2 fold. Conceivably, the noted mutation could affect the effectiveness of CFH in inhibiting the complement cascade. Other studies have explored the possible involvement of other complement pathway components, such as BF, C2, MBL2, and C7 in the etiology of AMD (Gold, Merriam et al. 2006), (Dinu, Miller et al. in press).
4. Smoking. Cigarette smoking is third only to older age and family history as a significant risk factor for AMD (Ambati, Ambati et al. 2003). One of the many negative effects of smoking is the activation of the complement system. Interestingly, one of the effects of cigarette smoke is to decrease the ability of CFH to bind and inhibit C3 (Kew, Ghebrehiwet et al. 1985).
5. Local source of active complement components. Significant amounts of C3, C5, C9 (Mullins, Russell et al. 2000), vitronectin (Hageman, Mullins et al. 1999), and clusterin (Wong, Pfeffer et al. 2000) mRNAs are present in the neural retina, RPE/choroid, and/or in cultured human RPE cells. Similarly, virtually all of the complement components are expressed by neurons (Yasojima, Schwab et al. 1999). Furthermore, mRNA levels for complement proteins seem to be significantly upregulated in affected areas of AD brain(Yasojima, Schwab et al. 1999). An increase in the concentrations of mRNAs encoding the complement proteins, as well as in the proteins themselves, have also been associated with atherosclerotic plaques (Yasojima, Schwab et al. 2001; Yasojima, Schwab et al. 2001). Thus, production and activation of the complement cascade may be a local phenomenon, occurring without the need of blood-borne elements. In contrast, it has been noted that some complement regulatory components are not upregulated locally. For example, in atherosclerotic plaques, there is no up-regulation in the synthesis of the defensive proteins C1 inhibitor, decay accelerating factor, CD46, C4 binding protein, or of the MAC inhibitor CD59 (Yasojima, Schwab et al. 2001). Similarly, C1 inhibitor and CD59 are not upregulated in AD brain (while active complement components are) (Yasojima, McGeer et al. 1999).
6. MAC presence in various drusen-exhibiting diseases. Western blot analysis found the assembled MAC complex in brain extracts from AD but not normal brains (Yasojima, Schwab et al. 1999). The MAC also has been observed on the surface of damaged host cells in myocardial infarct (Yasojima, Schwab et al. 1998) and atherosclerotic plaques (Yasojima, Schwab et al. 2001; Yasojima, Schwab et al. 2001). Similarly, the MAC is considered the principal injury cause in some forms of glomerulonephritis: “the principal mediator of immune complex-mediated glomerular injury is the complement system, especially C5b-9 membrane attack complex formation” (Nangaku and Couser 2005). I extend this model to AD and AMD by identifying the MAC (C5-9) formation as the principal cause of disease.
7. AMD mouse model. Ambati et al (Ambati, Anand et al. 2003) recently proposed a mouse model for AMD in which the mechanism of attracting macrophages to complement residue deposition was impaired. The authors observed that mice deficient in Ccl-2 (monocyte chemoattractant protein-1) or its cognate receptor Ccr2 (C—C chemokine receptor-2), which are involved in recruiting macrophages to loci of complement opsonization, present features characteristic of human AMD, such as drusen accumulation and lipofuscin deposits in “swollen and vacuolated RPE cells” (Ambati, Anand et al. 2003). This observation concurs with my model in which I propose that the gradual accumulation of complement debris at a rate that exceeds the rate of debris removal, e.g. by macrophages, causes the development of AMD.
8. Role of amyloid beta secondary to MAC-laden debris elimination in drusen formation and disease. Amyloid plaques (ak.a. senile plaques), neurofibrillary tangles and neuronal degeneration have constituted the hallmarks of AD for a century. The amyloid plaque deposits in AD are also riddled with a broad spectrum of inflammatory mediators, including complement components (McGeer and McGeer 2002). Recently, amyloid beta deposits have also been found in AMD drusen (Johnson, Leitner et al. 2002; Dentchev, Milam et al. 2003). Interestingly, amyloid beta deposits were not found in drusen of normal eyes, and were found with predilection in the eyes of patients with medium to heavy drusen formations, suggesting that amyloid-beta deposits might be associated with advanced stages of AMD (Dentchev, Milam et al. 2003). Since amyloid beta can activate the complement system (Bradt, Kolb et al. 1998), it is generally considered that inflammation in AD is a secondary pathologic effect, following the primary effect of amyloid plaques formation (Akiyama, Barger et al. 2000; McGeer and McGeer 2002). Contrary to this view, I propose here that complement activation, MAC formation and drusen deposits precede the formation of amyloid beta plaques in AD brains. I propose that amyloid beta gradually accumulates around drusen “seeds” formed by complement-induced cellular debris. For example, it is known that amyloid beta can form complexes with complement components such as C1q (McGeer and McGeer 2002) or activation products of C3 (Bradt, Kolb et al. 1998). A model with my proposed chronological order, in which amyloid beta starts accumulating around existing complement-derived drusen, could explain findings that Johnson et al (Johnson, Leitner et al. 2002) left unexplained—the fact that spherical deposits of amyloid-beta in eye had a iC3b-rich inner sphere: “the significance of the iC3b-rich inner sphere has not been established” (Johnson, Leitner et al. 2002). The model presented here, where amyloid-beta accumulates around “seeds” of complement-generated cellular debris, can also account for observations from another study where no amyloid-beta deposits were found in the drusen from the eyes of normal patients, but 4 out of 9 patients with AMD had amyloid-beta deposits (Dentchev, Milam et al. 2003). Moreover, “within these eyes, Aβ localized to a subset of drusen . . . Many drusen in normal and AMD eyes lacked Aβ positive vesicles, so the vesicles are not a consistent component of drusen” (Dentchev, Milam et al. 2003). It has not escaped my notice that the model presented here reverses the current dogma surrounding the etiology of AD, which attributes a primary causal and pathologic role to amyloid beta plaques. My model challenges this view by attributing the primary role to the formation of complement-driven cellular debris around which amyloid beta plaques later form.
9. Epidemiologic association between drusen-related diseases. Various studies have found associations between AMD and AD (Klaver, Ott et al. 1999), AMD and atherosclerosis (Vingerling, Dielemans et al. 1995), atherosclerosis and AD (Hofman, Ott et al. 1997; Roher, Esh et al. 2003). These results are consistent with the possibility of common disease pathways. The model presented here identifies the gradual creation of drusen through the elimination of cellular debris from cells affected by sub-lethal complement attack, followed by the gradual activation of multiple immune-related responses and inflammation. The association between these diseases affecting different tissues and organs could be explained by an over-active complement system at the entire organism level, due to genetic background, age, environmental factors, chronic inflammation, etc.

Etiology of Drusen and the Pathologic Role of Drusen

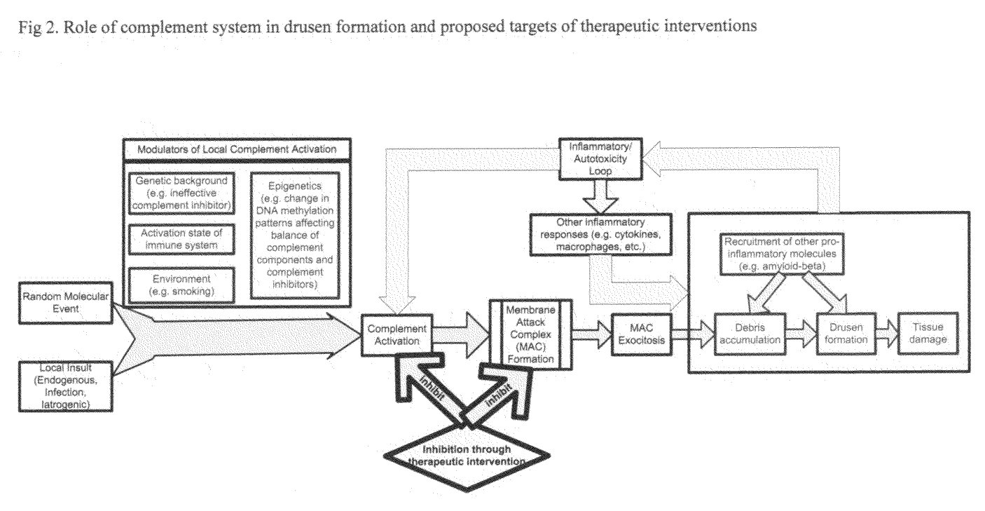

The complement activation the leads to AMD or AD is not an acute pathological event. A lethal acute response of the complement system would cause a rapid destruction of the targeted cells or tissues in a matter of hours or days, as is the case with the response of the complement system during a pathogenic infection. Rather, AMD and AD are diseases that progress slowly, over the course of years and decades. The local activation of the complement can be just a random molecular event, happening at a relatively low rate; the rate of activation could be modulated by various environmental factors, such as smoking, genetic or epigenetic factors, or general activation state of the immune system (FIG. 2). The full activation of the complement system, resulting in the formation of the MAC, can lead to exocitosys of the MAC together with other cellular debris that can be rich in complement components, such as products of C3 processing. The creation of debris that follows the activation of the complement system, coupled with its gradual accumulation in extra-cellular spaces at a rate that exceeds the rate at which it is removed, e.g. by macrophages, provides the “seeding” process through which drusen starts to accumulate. Gradually, other molecules (such as amyloid-beta, apoE, etc.) are recruited and the drusen start growing in size and quantity. A feedback pro-inflammatory cycle is generated where the existing cellular debris engages other immune components, such as dendritic cells, neutrophils, macrophages, as well as further activation of the complement. The resulting inflammation can affect the surrounding tissues (e.g. photoreceptors and RPE in the case of AMD, or neurons in the case of AD) and lead to disease.

Implications for Drug Based Therapies

Consistent with the model presented here for drusen formation and disease etiology, I propose that inhibiting the activation of the complement pathway, and especially the formation of the terminal MAC, as therapeutic strategies for countering the accumulation of extra-cellular debris and its pathologic role in diseases such as AMD, AD, and other diseases involving the gradual accumulation of extra-cellular debris, such as atherosclerosis. To achieve the prevention and/or cure of the disorders, one ore more inhibitors of the complement pathway can be used, such as small molecules, antibodies, proteins or peptides, microRNAs and siRNAs.

It would be preferable that the inhibition of the complement cascade would be early/preventive (e.g., before the full onset of a heterogeneous immune response and inflammation that would be harder to control), local (e.g., targeted for the eye, or even RPE, in the case of AMD), and modulated to the individual's needs (e.g., based on the general activation state of the immune system in a certain individual, based on genetic and epigenetic characteristics, age, etc). Alternatively, the inhibition of the complement cascade can be organism wide or localized to the affected and surrounding tissues.

The inhibition can be performed at any stage of the complement cascade, or focused on a particular step, such as the inhibition of the complement terminal membrane attack complex (MAC), formed by proteins C5b, C6, C7, C8, and multiple C9.

### Preferred Embodiment

A preferred embodiment would be the use of a MAC inhibitor, such as CD59 or CD59-like protein or peptide, as previously proposed in U.S. Pat. No. 5,573,940 with the aim of protecting transplanted organs and cells from cytolysis. Such an inhibitor would be delivered directly to the tissue involved in the disorder, e.g. brain tissue in AD or eye tissue in AMD. The administration would occur in individuals with an early stage of the respective disorder (e.g., diagnosed via imaging of early extra-cellular deposits in the vicinity of the affected tissue, or through the use of antibodies) or in individuals without exhibiting the disease but at higher risk of developing the disease based on risk assessment (e.g., genomic risk factor or biomarker presence). The amount of inhibitory substance would be determined based on the stage of the disease and on the general state of activation of the complement system in the respective individual, or based on dosage determined to be efficient by the composition developer.

### Additional Embodiments

Additional embodiments of the method for treating AMD, AD, and other diseases involving the gradual accumulation of extra-cellular debris, such as atherosclerosis, could involve the use of one or more of the following complement inhibitors:
1. Alternative MAC inhibitors (miRNA or siRNA, small molecules, antibodies, proteins or peptides inhibiting one of the MAC components: C5b, C6, C7, C8 or C9 complement components)
2. C5 inhibitors (miRNA or siRNA, small molecules, antibodies, proteins or peptides). An example of a CD59-like inhibitor is eculizumab, developed by Alexion (New Haven, Conn.), a humanized monoclonal antibody that specifically blocks cleavage of the C5 component of the complement system and prevents the generation of the potent anaphylatoxin C5a and the cytolytic C5b-9 complex. Eculizumab is a long-acting, humanised version of the anti-C5 antibody. The short-acting version is called pexelizumab. Eculizumab has been assessed for its efficacy in treating paroxysmal nocturnal haemoglobinuria, asthma and transplantation. I propose here that Eculizumab and/or pexelizumab could be used for treatment and/or prevention of AMD, AD, and other diseases involving the gradual accumulation of extra-cellular debris, such as atherosclerosis
3. C3 inhibitors (miRNA or siRNA, small molecules, antibodies, proteins or peptides). An example includes compstatin, a synthetic 13 amino acid cyclic peptide, developed by University of Pennsylvania and investigated for transplant, strokes, heart attacks, and burn injuries.
4. C1 inhibitors (miRNA or siRNA, small molecules, antibodies, proteins or peptides). An example includes Berinert P developed by Aventis, whose use has not been proposed for AD, AMD, or atherosclerosis.
5. APT070 (Mirococept), a membrane-localised complement inhibitor based on a recombinant fragment of soluble CR1, developed by Inflazyme Pharmaceuticals and currently under investigation for treatment of rheumatoid arthritis and intestinal ischaemia and reperfusion injury.
6. ETI-201, an anti-CR1 antibody crosslinked to double stranded DNA, developed by Elusys and investigated for the treatment of lupus.

Additional embodiments of the method for treating AMD, AD, and other diseases involving the gradual accumulation of extra-cellular debris, such as atherosclerosis, could involve the use of one or more of the following delivery methods
1. orally
2. intravenously
3. topical application
4. intravitreously

Additional embodiments of the method for treating AMD, AD, and other diseases involving the gradual accumulation of extra-cellular debris, such as atherosclerosis, could involve the use of one or more of the following localization methods:
1. whole body inhibition of complement activity
2. localized (e.g., tissue, organ) inhibition of complement activity

Additional embodiments of the method for treating AMD, AD, and other diseases involving the gradual accumulation of extra-cellular debris, such as atherosclerosis, could involve the inhibition of the complement pathway in:
1. individuals who have already developed the disorder
2. individuals who exhibit early signs of the disorder
3. individuals deemed at risk of developing the disorder

### CONCLUSION

To achieve the prevention and/or cure of AMD, AD, and other diseases involving the gradual accumulation of extra-cellular debris, one ore more inhibitors of the complement pathway can be used, such as small molecules, antibodies, proteins or peptides, microRNAs and siRNAs.

It would be preferable that the inhibition of the complement cascade would be early/preventive (e.g., before the full onset of a heterogeneous immune response and inflammation that would be harder to control), local (e.g., targeted for the eye, or even RPE, in the case of AMD), and modulated to the individual's needs (e.g., based on the general activation state of the immune system in a certain individual, based on genetic and epigenetic characteristics, age, etc). Alternatively, the inhibition of the complement cascade can be organism wide or localized to the affected and surrounding tissues.

The inhibition can be performed at any stage of the complement cascade, or focused on a particular step, such as the inhibition of the complement terminal membrane attack complex (MAC), formed by proteins C5b, C6, C7, C8, and multiple C9.

Although preferred and other embodiments of the invention have been described herein, further embodiments may be perceived by those skilled in the art without departing from the scope of the invention as defined by the following claims.

What is claimed is:

1. A method for treating and/or preventing disorders that exhibit extra-cellular debris deposits in the vicinity of the affected tissues comprising administering an effective amount of a pharmaceutical composition comprising one or more agents that inhibit the complement pathway.

2. The method in claim 1 wherein the inhibiting agent is a small molecule.

3. The method in claim 1 wherein the inhibiting agent is an anti-sense oligonucleotide, miRNA, or siRNA.

4. The method in claim 1 wherein the inhibiting agent is an antibody.

5. The method in claim 1 wherein the inhibiting agent is a protein or peptide.

6. The method in claim 1 wherein the inhibiting agent is an antagonist.

7. The method in claim I wherein the inhibiting agent inhibits the gene expression of a complement pathway component.

8. The method in claim 1 wherein the said disorder is age related macular degeneration.

9. The method in claim 8 wherein the inhibiting agent is eculizumab, pexelizumab, compstatin, Berinert P, Mirococept, ETI-201 or a combination thereof.

10. The method in claim 1 wherein the said disorder is Alzheimer's disease.

11. The method in claim 10 wherein the inhibiting agent is eculizumab, pexelizumab, compstatin, Berinert P, Mirococept, ETI-201 or a combination thereof.

12. The method in claim 1 wherein the said disorder is atherosclerosis.

13. The method in claim 13 wherein the inhibiting agent is eculizumab, pexelizumab, compstatin, Berinert P, Mirococept, ETI-201 or a combination thereof.

14. The method in claim 1 wherein the inhibiting agent inhibits the C3, C5, C6, C7, C8, C9 component of the complement pathway or a combination thereof.

15. The method in claim 1 wherein the inhibiting agent inhibits the formation of the terminal complement membrane attack complex.

16. The method in claim 1 wherein the inhibiting agent is eculizumab, pexelizumab, compstatin, Berinert P, Mirococept, ETI-201 or a combination thereof.

17. The method in claim 1 wherein the inhibition of the complement pathway is localized to the tissues affected by the disorder.

18. The method in claim 1 wherein the inhibition of the complement pathway is at the level of the entire organism.

19. A method for treating and/or preventing Age Related Macular Degeneration comprising administering an effective amount of a pharmaceutical composition comprising eculizumab, pexelizumab, compstatin, Berinert P, Mirococept, ETI-201 or a combination thereof.

20. A method for treating and/or preventing Alzheimer's disease comprising administering an effective amount of a pharmaceutical composition comprising eculizumab, pexelizumab, compstatin, Berinert P, Mirococept, ETI-201 or a combination thereof.
